(19) United States
(12) Patent Application Publication
(10) Pub. No.: US 2023/0142909 A1
(43) Pub. Date: May 11, 2023

(54) CLINICALLY MEANINGFUL AND PERSONALIZED DISEASE PROGRESSION MONITORING INCORPORATING ESTABLISHED DISEASE STAGING DEFINITIONS

(71) Applicant: KONINKLIJKE PHILIPS N.V., EINDHOVEN (NL)

(72) Inventor: Claire Yunzhu ZHAO, BOSTON, MA (US)

(21) Appl. No.: 17/917,577

(22) PCT Filed: Apr. 10, 2021
(86) PCT No.: PCT/EP2021/059361

§ 371 Date: Oct. 7, 2022

Related U.S. Application Data
(60) Provisional application No. 63/008,082, filed on Apr. 10, 2020.

(51) Int. Cl. G16H 50/30

(52) U.S. Cl. CPC G16H 50/30

(57) ABSTRACT

A non-transitory computer readable medium (26) stores instructions executable by at least one electronic processor (20) to perform a method (100) for staging a disease having a predefined ordered set of S discrete stages where S is an integer having a value greater than or equal to two. The method includes: for each discrete stage of the S discrete stages, defining a representative vector (30) for the discrete stage in a vector space defined by a set of clinical metrics based on a set of training patients labeled with the discrete stage and with values for the set of clinical metrics; for a patient to be staged, receiving patient values for the set of clinical metrics; generating at least one stage value for the patient to be staged based on distances in the vector space between a patient vector defined in the vector space by the patient values for the set of clinical metrics and the representative vectors for the S discrete stages in the vector space; and displaying the at least one stage value for the patient to be staged on a display device (24) operatively connected with the electronic processor.

### FIELD

The following relates generally to the disease staging arts, disease progression monitoring arts, patient monitoring arts, and related arts.

### BACKGROUND

Disease staging assigns a clinically based measure of severity for a patient using medical criteria to assess a stage of disease progression. A disease staging process comprises a classification system that uses diagnostic findings to classify patients. The stages of the staging system are designed based on “first principles” clinical considerations to group patients who require similar treatment and have similar expected outcomes into a given stage. Typically, the stages are defined in terms of a small number of readily assessed clinical metrics that have identifiable associations to the disease. For example, acute kidney disease (AKI) is commonly staged based on two clinical metrics: serum creatinine level and urine output. Creatinine is removed from the blood by operation of the kidneys—hence, elevated serum creatinine is associated with poor kidney function. Urine is generated by the kidneys—hence, reduced urine output is also associated with poor kidney function. Usually, the staging system defines an ordered set of stages, where the ordering of the stages corresponds to progressive worsening of the disease. The number of stages in a staging system is usually small (i.e., the staging system is coarse), e.g. AKI staging uses three or four stages.

Finer grading would have certain benefits. Finer grading could provide for better assessment of the patient and for earlier intervention. A finer disease grading system could also be more effectively used for monitoring purposes, such as in pinpointing medication dosages and assessing treatment effectiveness. However, a finer disease grading system having a larger number of stages typically results in the stages not having a readily identifiable association to the disease, and hence lack clinical interpretation. That is, it can be challenging to assign physiological or pathophysiological meanings to the stages. As a result, these models are difficult to be adapted to the current clinical workflow. Moreover, existing disease staging systems were often developed by professional medical associations or the like based on extensive study of the relevant clinical literature, and have become consensus disease staging systems that are widely adopted by hospitals and familiar to clinicians. It is difficult to then discard an existing, widely adopted disease staging for a new disease grading system, especially one whose finer-grained stages may be less readily associated to the disease on a clinical first principles basis. Furthermore, developing a new disease grading system usually requires extensive domain-specific knowledge about the disease, which may be unavailable in many contexts such as an individual hospital or hospital department.

The following discloses certain improvements to overcome these problems and others.

### SUMMARY

In one aspect, a non-transitory computer readable medium stores instructions executable by at least one electronic processor to perform a method for staging a disease having a predefined ordered set of S discrete stages where S is an integer having a value greater than or equal to two. The method includes: for each discrete stage of the S discrete stages, defining a representative vector for the discrete stage in a vector space defined by a set of clinical metrics based on a set of training patients labeled with the discrete stage and with values for the set of clinical metrics; for a patient to be staged, receiving patient values for the set of clinical metrics; generating at least one stage value for the patient to be staged based on distances in the vector space between a patient vector defined in the vector space by the patient values for the set of clinical metrics and the representative vectors for the S discrete stages in the vector space; and displaying the at least one stage value for the patient to be staged on a display device operatively connected with the electronic processor.

In another aspect, an apparatus for staging a disease having a predefined ordered set of S discrete stage where S is an integer having a value greater than or equal to two includes at least one electronic processor. A non-transitory computer readable medium stores instructions readable and executable by at least one electronic processor to perform a method including: for a patient to be staged, receiving patient values for a set of clinical metrics; using the received patient values, defining a patient vector in a vector space defined by the set of clinical metrics; generating at least one stage value for the patient to be staged based on distances in the vector space between the patient vector and representative vectors in the vector space that represent respective discrete stages of the predefined ordered set of S discrete stages; and controlling a display device operatively connected with the electronic processor to display the at least one stage value for the patient to be staged.

In another aspect, a method for staging a disease having a predefined ordered set of S discrete stages where S is an integer having a value greater than or equal to two includes: for each discrete stage of the S discrete stages, defining a representative vector for the discrete stage in a vector space defined by a set of clinical metrics based on a set of training patients labeled with the discrete stage and with values for the set of clinical metrics by operations including: defining training patient vectors in the vector space corresponding to the respective training patients labeled with the discrete stage by the values for the set of clinical metrics labeling the respective training patients; and defining the representative vector for the discrete stage in the vector space as a centroid of the constructed training patient vectors in the vector space; for a patient to be staged, receiving patient values for the set of clinical metrics; generating at least one stage value for the patient to be staged based on distances in the vector space between a patient vector defined in the vector space by the patient values for the set of clinical metrics and the representative vectors for the S discrete stages in the vector space; and displaying the at least one stage value for the patient to be staged on a display device.

One advantage resides in providing a disease staging process that provides finer-grained staging while retaining the stages of a pre-existing disease staging system.

Another advantage resides in providing a disease staging process that provides a detailed description for each disease without including overwhelming details to makes the disease staging process cumbersome.

Another advantage resides in providing a disease staging process that maps to an existing disease staging classification so that clinicians do not have to adopt an entirely different grading system, which will ease burden of interpretation and adhere to existing established guidelines and staging systems published by professional societies.

Another advantage resides in providing a disease staging process that is able to produce finer grains in disease staging, and number of sub-classes is adjustable to user needs.

Another advantage resides in providing a disease staging process that does not require the complicated domain knowledge for finer staging of each specific disease, which is not disease-specific and can be directly applied to all diseases.

Another advantage resides in providing a disease staging process that can easily be updated if clinical definitions for the disease in question is revised.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for disease staging in accordance with the present disclosure.

FIGS. 2 and 3 show exemplary flow chart operations performed by the apparatus of FIG. 1.

FIGS. 4 and 5 diagrammatically illustrate representative vectors in vector space generated by the apparatus of FIG. 1.

FIG. 6 shows an example output by the apparatus of FIG. 1.

FIG. 7 shows an example of possible treatments assigned to different levels of outputs by the apparatus of FIG. 1.

### DETAILED DESCRIPTION

Existing disease staging usually employs a coarse ordered set of stages defined by staging criteria that use only a few clinical metrics. For example, acute kidney injury (AKI) staging employs four stages (“Stage 0 or no AKI”, “stage 1”, “stage 2”, and “stage 3”), with the staging criteria being defined in terms of clinical metrics, including serum creatinine and urine output. The stages are ordered in the sense that there is a defined progression of stages indicating increasing seriousness of the disease (e.g., as measured by clinical considerations such as more debilitating, higher risk of death, higher risk of triggering clinical complications, and/or so forth). In the AKI example, “stage 1” is a more serious stage of AKI compared with “stage 0”; “stage 2” is a more serious stage of AKI compared with “stage 1”; and “stage 3” is a more serious stage of AKI compared with “stage 2”. A patient with increasingly worsening AKI thus progresses through the ordered set of stages from “stage 0” to “stage 1” to “stage 2” to “stage 3”. This type of conventional staging is easily performed manually since it utilizes only a few clinical metrics and is usually defined by a deterministic algorithm. The stages are familiar to clinicians and it is easy for clinicians to understand the staging criteria at a first principles level. However, conventional staging provides limited information for clinical decision making.

The following discloses a staging approach for providing finer-grained (e.g. continuous) staging, while still retaining the standard clinical stages. Further the staging approach can be readily implemented automatically, without detailed understanding of the expert domain of the disease being staged, and optionally retains the staging nomenclature of the pre-existing coarse disease grading system.

The disclosed staging approaches employ a training set of patients, in which each training patient is labeled by various clinical metrics preferably (but not necessarily) including the clinical metrics used in the conventional staging (e.g., serum creatinine and urinary output in the case of AKI) but also including other clinical metrics. The training patients are also labeled as to stage using the conventional staging criteria. This stage labeling can be done manually, or using a deterministic algorithm if the conventional staging is available as an algorithm.

For each labeled stage, a representative patient is identified in the vector space defined by the set of clinical metrics. An approach for identifying a representative patient for a given stage is to take the centroid of all patients labeled with that stage, optionally after removing any obvious outliers. In this approach, the representative patient is a construct, not necessarily one of the training patients. Hence, each stage is identified by the location of the representative patient in the vector space, that is, by a representative vector in the vector space.

When staging a new patient (not part of the training set), the clinical metrics defining the vector space are measured for the new patient and the patient's location in the vector space is thus defined, that is, a patient vector is defined in the vector space. Coarse staging can be done by computing the distances between the patient's location and the representative patients of the stages (that is, the distances between the patient vector and each of the representative vectors), and selecting the closest stage as the stage of the new patient. A more precise (e.g. continuous) stage can additionally or alternatively be computed from the projection of the stage-to-patient vector onto a current stage-to-next stage vector.

Advantageously, the accuracy of the disclosed staging approach as compared with the conventional staging is easily quantified by checking whether the coarse stage output by the disclosed approach matches the coarse stage obtained by the standard staging criteria. Accuracy of the precise (e.g. continuous) staging can be assessed quantitatively based on the component of the stage-to-patient vector that is orthogonal to the stage-to-next stage vector (where a smaller orthogonal component implies more accuracy).

Patient staging can be done using the disclosed approach as frequently as needed. For some diseases such as AKI, it is contemplated to update the AKI stage in real-time based on the latest patient data acquired (in part) by continuous patient monitoring.

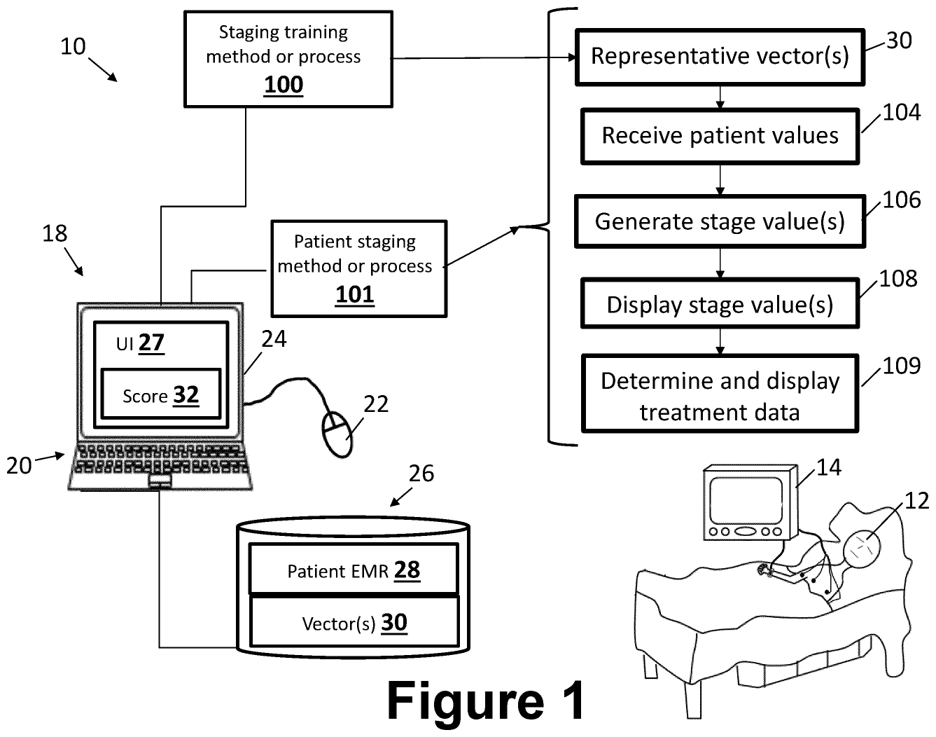

With reference to FIG. 1, an illustrative apparatus 10 is shown for a disease staging process. The illustrative example provides disease staging for a hospitalized patient 12 monitored by a bedside patient monitor 14. FIG. 1 also shows an electronic processing device 18, such as a workstation computer, nurses' station computer or electronic whiteboard, tablet, phone, the illustrative patient monitor 14, various combinations thereof, or more generally a computer. The electronic processing device 18 may also include a server computer or a plurality of server computers, e.g. interconnected to form a server cluster, cloud computing resource, or so forth, to perform more complex computational tasks. The computer 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices (e.g., a first display for inputting patient parameters or clinical metrics, and a second display for showing a disease acuity score).

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 27 for display on the display device 24.

The apparatus 10 is configured as described above to perform a staging training method or process 100 for generating a disease staging system for a disease that is based on a predefined ordered set of S discrete stages (where S is an integer having a value greater than or equal to two). The apparatus 10 is further configured as described above to perform a patient staging method or process 101 for generating at least one stage value 32 for the patient 12 to be staged using the disease staging system trained by the stage training method or process 100. The non-transitory storage medium 26 stores instructions which are readable and executable by the at least one electronic processor 20 to perform disclosed operations including performing the methods or processes 100, 101. The disease staging systems and method disclosed herein consume clinical metrics such as laboratory tests (e.g. blood test results), periodic patient measurements (e.g. urinary output), continuously monitored vital signs (e.g., heart rate, respiratory rate, SpO₂, and/or so forth acquired for the patient 12 using the patient monitor 14), various combinations thereof, and/or so forth. The patient data is typically stored in a patent electronic medical record (EMR) 28 on the non-transitory storage medium 26 and retrieved therefrom when performing the patient staging method or process 101. In some examples, the staging training method 100 is computationally complex, and may be advantageously performed at least in part by cloud processing. On the other hand, the patient staging method or process 101 is generally less computationally complex, and may be performed by a nurses' station computer, the patient monitor 14, or the like. This is merely a non-limiting example.

With continuing reference to FIG. 1, an illustrative embodiment of the disease staging processing 100, 101 is diagrammatically shown as a flowchart. The at least one electronic processor 20 is programmed to perform the disease staging method or process 100 to generate a representative vector 30 for each respective discrete stage of the S disease stages of a pre-defined (i.e. existing) conventional disease staging system. Using the conventional AKI staging system as an example, the disease staging method or process 100 generates a representative vector for each of the “high risk”, “stage 1”, “stage 2”, and “stage 3” stages of the conventional AKI staging system. The representative vectors 30 are defined in a vector space defined by a set of clinical metrics based on a set of training patients labeled with the discrete stage and with values for the set of clinical metrics. It should be noted that while the vector space is defined by the set of clinical metrics, the dimensions of the vector space may not be identical with the metrics of the set of clinical metrics. For example, the set of clinical metrics may be processed by Principal Component Analysis (PCA) and the vector space may then be defined by the top two (or three, or four, et cetera) principal components generated by the PCA. In generating the training data, the discrete stage labels may be automatically assigned to the training patients using a deterministic staging algorithm based on values of a subset of the set of clinical metrics, or may be assigned manually by clinicians (e.g., assigned by the training patients' respective doctors and extracted from the records for the training patients stored in the EMR 28). The deterministic staging algorithm assigns a discrete stage selected from the predefined ordered set of S stages.

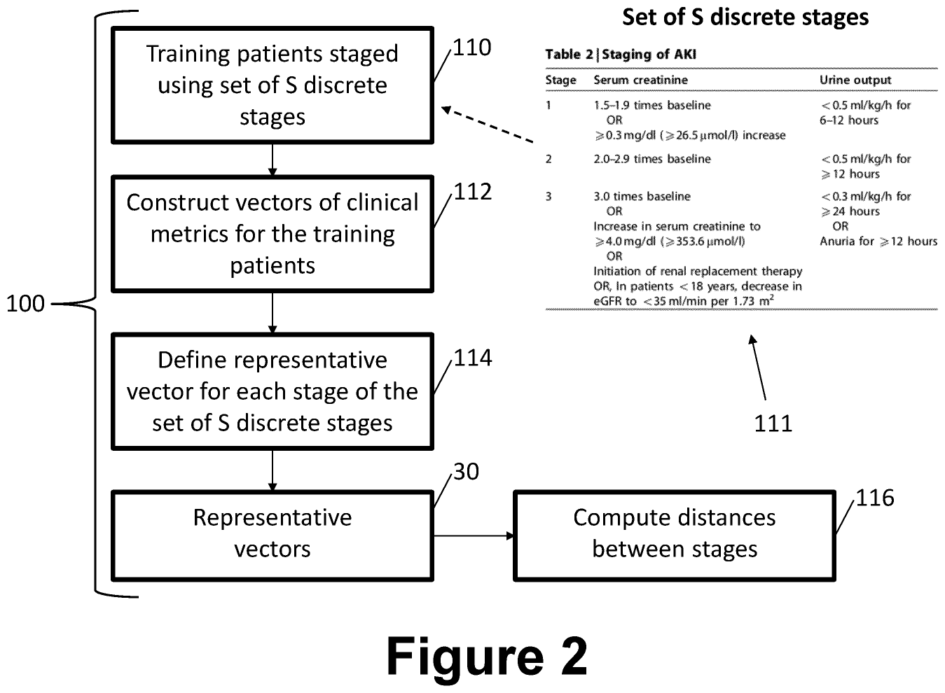

With brief reference to FIG. 2, to define the representative vectors 30, in an operation 110 the training data set of training patients is created, with the training patients staged using the ordered set of S discrete stages of the conventional staging system. Illustrative FIG. 2 is described with reference to training an AKI staging system, and uses a non-limiting illustrative example 111 of a set of S discrete stages for an existing AKI staging system. In this illustrative example, the ordered set of S discrete stages 111 includes “Stage 0”, “Stage 1”, “Stage 2”, and “Stage 3”, with “Stage 0” being the least serious stage, “Stage 3” being the most serious stage, and “Stage 1” and “Stage 2” being intermediate between “Stage 0” and “Stage 3”. “Stage 0” corresponds to a patient who has not yet been diagnosed with AKI but is at risk for such a diagnosis. In the operation 110, the training patients are labeled with respective stages by applying the staging criteria of the illustrative example 111 or by retrieving physician-assigned stages for the training patients from the EMR 28. Additionally, the values of a set of clinical metrics are collected for each training patient. Typically, the set of clinical metrics is a superset of the clinical metrics used in the conventional staging system 111. For the AKI example, the set of clinical metrics thus includes the serum creatinine and urinary output clinical metrics, and additionally includes other clinical metrics such as, by way of non-limiting illustrative example, one or more of: other bloodwork results (e.g. white blood cell count, red blood cell counts, etc.), vital signs (e.g. heart rate, respiratory rate, etc.), other recorded patient data such as a patient consciousness assessment (e.g. using the standard AVPU scale), various combinations thereof, and/or so forth. Optionally, if the values for a small number of the clinical metrics are unavailable for a particular training patient then that patient may be assigned the average value for that metric of all other patients of the stage in the training set.

In an operation 112, training patient vectors corresponding to the respective training patients are constructed in a vector space defined by the set of clinical metrics. The vector space may be directly defined by the clinical metrics, i.e. each clinical metric may be a dimension of the vector space. In this approach, if there are N clinical metrics then the vector space would have N dimensions corresponding to the N clinical metrics. Alternatively, the vector space can be defined by the clinical metrics by, for example, applying Principal Component Analysis (PCA) to the values of the clinical metrics for the patients of the training set and the vector space may then be defined by the top two (or three, or four, et cetera) principal components generated by the PCA. Each training patient vector is labeled with the discrete stage assigned to that training patient in the operation 112.

In an operation 114, the representative vector 30 is defined in the vector space for each stage of the ordered set of S stages 111. In one suitable approach, for each stage of the conventional staging system 111, the corresponding representative vector 30 is defined as a centroid of the training patient vectors in the vector space that are labeled by that stage. For the AKI example, the representative vector for “Stage 1” would be the centroid of all training patient vectors labeled with “Stage 1”; the representative vector for “Stage 2” would be the centroid of all training patient vectors labeled with “Stage 2”; the representative vector for “Stage 3” would be the centroid of all training patient vectors labeled with “Stage 3”; and (if used) the representative vector for “High risk” would be the centroid of all training patient vectors labeled with “High risk”. Optionally, the operation 114 may include additional or other processing for defining the representative vectors 30. For example, an outliers analysis may be performed on the training patient vectors for each stage and on any outliers removed prior to computing the centroid. Consistency checking may also be performed. For example, if the set of all training patient vectors labeled with a particular stage (after the optional outliers removal) is too spread-out, then the vector space may be redefined by refining the choice of the set of clinical metrics and/or repeating the PCA or other processing used in defining the vector space, to obtain a vector space in which the set of all training patient vectors labeled with the particular stage is acceptably compact.

In an optional operation 116, a maximum distance can also be defined between the centroids of the individual discrete stages. A suitable distance function can be selected to define the maximum distance, including a Euclidean distance function, a Hamming distance function, a Geometric distance function, a cosine distance function, and so forth. The distance function is selected as the one achieving a maximum distance between the centroids.

In some embodiments, the representative vectors 30 can be optimized by tailoring the operations 112 and/or 114. For example, a simulation of an occurrence of disease staging using simulated patient values can be performed. One or more key performance indicators (KPIs) can be calculated based on results of the simulation for the patient and the associated patient grading scores. The KPIs can include, for example, length of patient stay, utilization of invasive measure, and so forth on the patient. The representative vectors 30 can be adjusted if the KPIs fall below one or more predetermined quality thresholds. For instance, a correlation study can be performed between changes in a patient acuity score and an intervention process administered to the patient. If changes in the score based on the KPIs matches with the desired outcome of the intervention for patients with positive outcomes, then the design of the vector space and the choice of distance function used in optional operation 116 is validated; otherwise, an alternative distance function and design of the vector space (e.g., selection of different features as the dimensions of the vector space) needs to be selected. Additionally or alternatively, validation of the representative vectors 30 can be achieved through comparison against a clinical benchmark which is not commonly measured in the clinical setting. For example, if grading is performed on Acute Kidney Injury (AKI), this clinical benchmark could be the following biomarkers which have been scientifically found to improve risk stratification of AKI, but have not yet been widely measured in the clinics: urinary angiotensinogen (uAGT), urinary neutrophil gelatinase-associated lipocalin (uNGAL), and/or urinary IL-18 (uIL-18).

The staging training method or process 100 of FIGS. 1 and 2 is performed once to design the disease grading system in terms of the representative vectors 30, and thereafter may be used to grade the disease in various patients by applying the patient staging method or process 101. FIG. 1 diagrammatically shows operation of the patient staging method or process 101 for the illustrative patient 12. In an operation 104, patient values for the patient 12 to be staged are received for the set of clinical metrics.

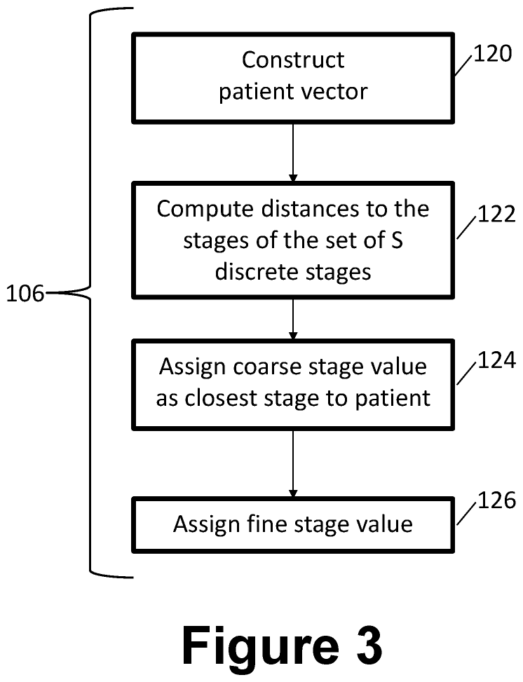

With continuing reference to FIG. 1 and with further reference to FIG. 3, at an operation 106, at least one stage value 32 is generated for the patient to be staged. As detailed in FIG. 3, in an operation 120 a patient vector is defined in the vector space (that is, the same vector space in which the representative vectors 30 are defined) by the patient values of the patient 12 to be staged for the set of clinical metrics. In an operation 122, distances are computed in the vector space from (or between) the patient vector and the representative vectors of the ordered set of S discrete stages of the conventional staging system 111. The distances can be determined using a distance function such as a Euclidean distance function, a Hamming distance function, a Geometric distance function, a cosine distance function, or so forth. In an operation 124 and/or an operation 126, the stage value(s) 32 are assigned based on the distances in the vector space between the patient vector and the representative vectors for the S discrete stages in the vector space computed in operation 122.

In one embodiment, an operation 124 is performed to generate a coarse stage value as the stage corresponding to the closest representative vector of the representative vectors 30 to the patient vector; that is, the coarse stage value is the stage for which the distance in the vector space between the patient vector and the corresponding representative vector is shortest. A coarse stage value for the patient to be staged is thus generated as the discrete stage represented by the closest representative vector.

In another embodiment, in addition or alternatively to identifying the closest representative vector of the representative vectors 30, a fine stage value is assigned. To do so, the two closest representative vectors of the representative vectors 30 for the S discrete stages that are closest to the patient vector are identified. The two closest representative vectors include a current stage representative vector corresponding to a current stage and a next stage representative vector corresponding to a next stage. The current stage is ordered lower than the next stage in the ordered set of S discrete stages. The fine stage value is generated for the patient 12 to be staged based on the current stage, the next stage, the distance in the vector space between the patient vector and the current stage representative vector, and the distance in the vector space between the patient vector and the next-stage representative vector.

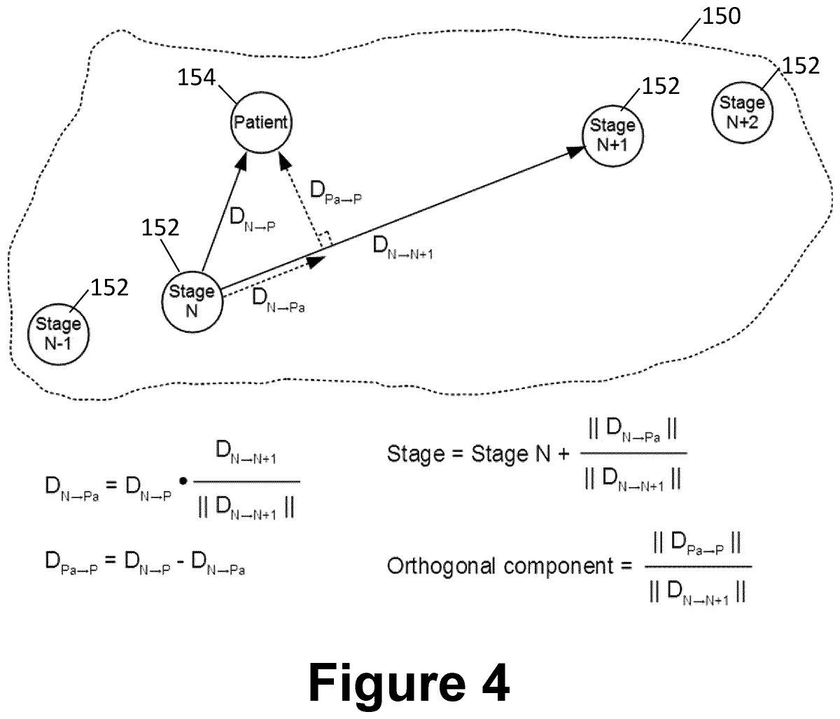

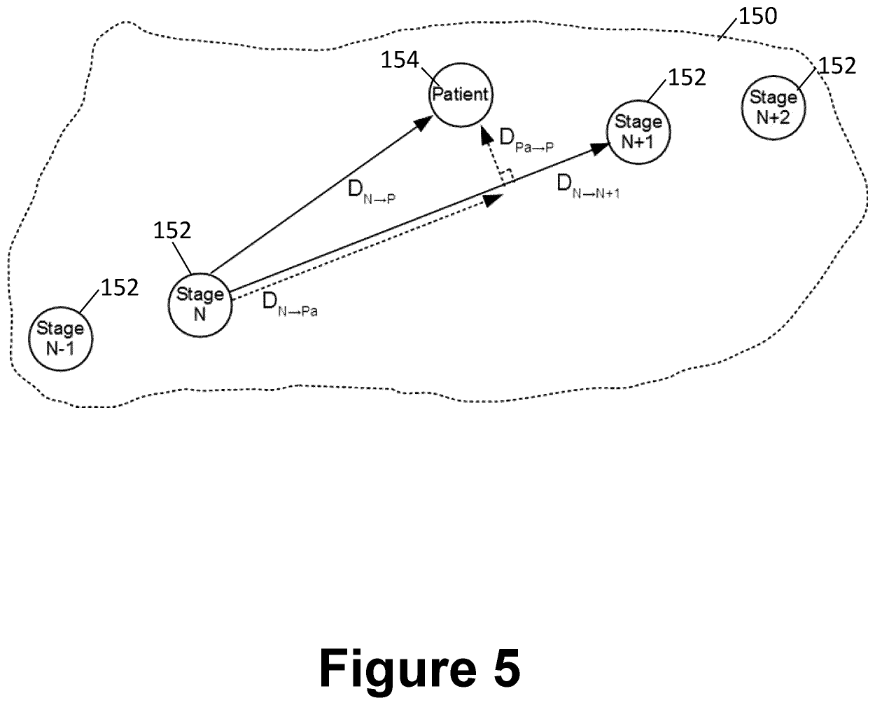

To further illustrate, FIGS. 4 and 5 show one suitable approach for the operation 126 of computing the fine stage value. FIGS. 4 and 5 diagrammatically indicate the vector space 150 (it should be understood that the vector space will typically have more than two dimensions, hence FIGS. 4 and 5 are diagrammatic). FIGS. 4 and 5 further illustrate four representative vectors 152 defined in the vector space 150 by the staging training method or process 100. Still further, FIGS. 4 and 5 illustrate a patient vector 154. In both the example of FIG. 4 and the example of FIG. 5, the two closest representative vectors to the patient vector 154 are representative vectors labeled as Stage N and a Stage N+1. In the example of FIG. 4, the representative vector for Stage N is closest to the patient vector 154; while, in the example of FIG. 5, the representative vector for Stage N+1 is closest to the patient vector 154. In both cases, Stage N is defined as the lower ordered stage of the two closest representative vectors in the ordered set of N stages, while Stage N+1 is defined as the higher ordered stage of the two closest representative vectors in the ordered set of N stages. A fine stage value for the patient 12 to be staged is generated based on the current stage (Stage N), the next stage (Stage N+1), the distance in the vector space between the patient vector and the current stage representative vector (denoted as vector D_{N→P}), and the distance in the vector space between the current stage representative vector and the next-stage representative vector (denoted as vector D_{N→N+1}). In one suitable notation for the fine stage value, the current stage is assigned a first integer value, the next stage is assigned a second integer value different from the first integer value, and the fine stage value is a real number lying between the first integer value and the second integer value. So, for the AKI example, if the current stage is “Stage 2” and the next stage is “Stage 3”, then the current stage can be assigned the integer value 2, the next stage can be assigned the integer value 3, and the fine stage value is a real number lying between 2 and 3.

As illustrated in FIGS. 4 and 5, in one example of this embodiment, the generating of the fine stage value is based on a variety of factors, including the current stage represented by the current stage representative vector, the next stage represented by the next stage representative vector, and a ratio of: (i) a length of a projection of a current stage-to-patient vector (defined as the vector starting at the current stage representative vector and ending at the patient vector) onto a current stage-to-next stage vector (defined as the vector starting at the current stage representative vector and ending at the next stage representative vector), and (ii) a length of the current stage-to-next stage vector.

With particular reference to FIG. 4, the generating of the fine stage value is based on the current stage, the next stage, and a ratio:

$$\frac{‖ {D}_{N \to P a} ‖}{‖ {D}_{N \to N + 1} ‖}$$

where ∥D_{N→N+1}∥ is the length of a vector D_{N→N+1} from the current stage representative vector to the next-stage representative vector, and D_{N→Pa} is a vector given by the dot product:

$${D}_{N \to P} \cdot \frac{{D}_{N \to N + 1}}{‖ {D}_{N \to N + 1} ‖}$$

where D_{N→P} is a vector from the current stage representative vector to the patient vector.

In other examples, the distance in the vector space between the patient vector and the representative vector can be linearized, which results in the at least one stage value 32 being directly linearly scaled to the distance between the centroid the closest representative vector and the second-closest representative vector.

With returning reference to FIG. 1, at an operation 108, the at least one stage value 32 for the patient to be staged is displayed on the display device 24. In some examples, the coarse stage value and/or the fine stage value can also be displayed on the display device 24. In some embodiments, the at least one stage value 32 is used to determine treatment data 109 for the patient 12. The treatment data 109 may comprise recommendations for performing varying types of intervention options to treat the patient. For example, the treatment data 109 may be a displayed recommendation to perform a specific pharmaceutical intervention or treatment (e.g., a medication or an intravenous (IV) drip), an imaging session (e.g., ultrasound (US), X-ray, magnetic resonance imaging (MRI), computed tomography (CT), and so forth), a non-imaging diagnostic (e.g., a blood test, a urine test, a pathology test such as a biopsy, etc.), a surgical intervention, or other form of intervention or treatment (e.g., invasive ventilation, cardiac assistive devices, organ transplant, etc.). In some examples, the medical professional may be required to provide an input to the computer 18 to control or otherwise order an intervention option. In another example, the computer 18 may be in communication with an associated drug intervention device to automatically commence a corresponding drug intervention session. In another example, the computer 18 may generate a medical imaging examination order, optionally including information such as imaging parameters for use in the imaging examination. The generated order may be automatically sent to a hospital radiology laboratory to schedule the imaging session, or may be sent to the patient's physician to review and issue the order. In another example, the computer 18 may generate a non-imaging diagnostic order, optionally including information such as diagnostic parameters.

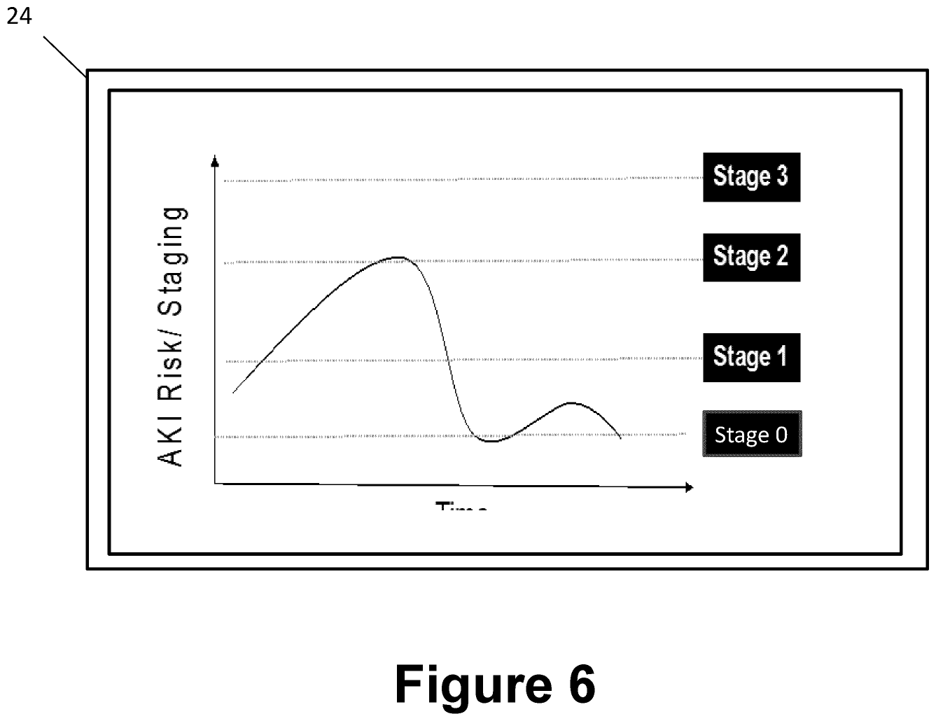

FIG. 6 shows an application of the patient staging method or process 101 in continuously staging the progression of AKI by showing the at least one stage value 32 displayed as a trendline over time. In particular, FIG. 6 shows an evaluation of treatment effectiveness via monitoring AKI using a standard clinical metric, such as Kidney Disease Improving Global Outcomes (KDIGO) stages (see, e.g., KDIGO Clinical Practice Guideline for Acute Kidney Injury. OFFICIAL JOURNAL OF THE INTERNATIONAL SOCIETY OF NEPHROLOGY, VOLUME 2, ISSUE 1, MARCH 2012). KDIGO criteria coarsely divides the progression of AKI in 4 stages, and only uses two clinical features (e.g., serum creatinine and urine output). FIG. 2 shows an example of finer grading of AKI, which also adheres to established guidelines. In this way, finer details and assessment of monitoring focuses and treatment categories can be achieved. Such a monitoring framework can be easily adapted to other diseases.

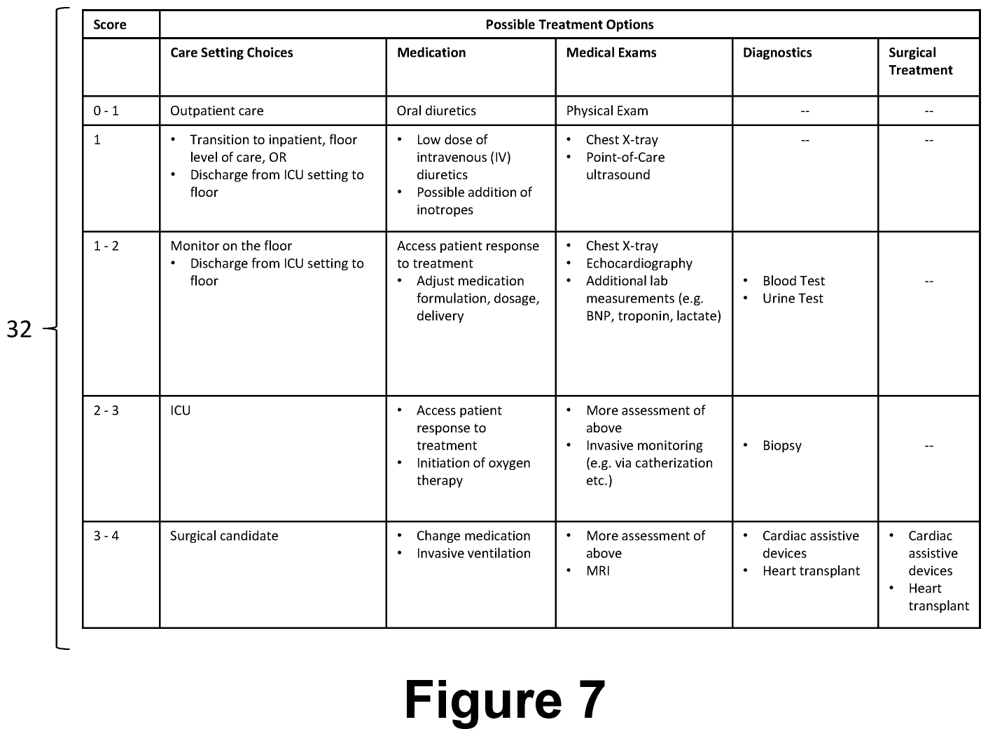

FIG. 7 shows an example of possible treatments or interventions assigned to different levels of acuity scores 32. As shown in FIG. 7, a range of acuity scores 32 can be separated into, for example, 5 levels of possible treatment levels. The 5 levels can include: acuity scores 32 ranging from 0-1; 1; 1-2; 2-3; and 3-4. It should be noted that while the illustrative example employs integer levels delineated by the S discrete stages, the levels could alternatively be delineated by continuous stage values generated as disclosed herein. FIG. 7 shows different medication, medical examination, diagnostics, or surgical treatments associated with each level. A higher level of acuity score is indicative of a higher level of intervention or treatment. For example, a score in the level of 0-1 can include oral diuretics or a physical examination, as opposed to a score in the level of 3-4, in which the interventions or treatments include changing medication, invasive ventilation, cardiac assistive devices, or a heart transplant.

While the apparatus 10 and the method 100, 101 are described primarily in terms of AKI, the disclosed apparatus and method can be used for staging other types of disease such as acute heart failure, in which established staging systems employing an ordered set of discrete stages is employed. In the case of acute heart failure, an established staging system is the Acute Decompensated Heart Failure National Registry (ADHERE) model, which uses as clinical metrics the blood urea nitrogen (BUN), systolic blood pressure (sBP), and serum creatinine. Alternatively, the heart failure classification identified by the New York Heart Association (NYHA), which uses signs and symptoms of fatigue and dyspnea at rest or in activity, may also be used as an established staging system to define the broad, coarse stages. The more precise, finer-grained staging can be useful in other scenarios, such as to assess a timing of hospitalization of the patient. With a more detailed grading, especially at early stages, hospitalization can be timelier before patient actually enters into the next disease stage, which is often already of high severity. In another example, resources need for patients can be analyzed for managing hospital resources, such as medication and inventory needs. In another example, a quality of patient care can be evaluated. Patients admitted to the hospital with advanced stages of illness may represent possible failures of outpatient care or care at the previous clinical settings. In a further example, clinical trials can be facilitated by providing a finer selection of patient cohorts, and a finer and more in-depth assessment of control vs. experimental groups.

Some examples of treatments or interventions have been provided herein solely for the purpose of illustration. One having ordinary skill in the medical arts would understand how the apparatus 10 and method 100,101 can, in some limiting embodiments, be implemented to provide appropriate or different treatment or intervention recommendations for different types of illnesses which are not described herein.

The apparatus 10 and the method 100, 101 can, in some non-limiting embodiments, be implemented as an improvement to existing commercial products that incorporate disease staging and/or early warning scoring, such as an Intellivue Guardian bedside monitor, or a Central Station (both available from Koninklijke Philips NV, the Netherlands), or any suitable electronic health record system.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method for staging a disease having a predefined ordered set of S discrete stages where S is an integer having a value greater than or equal to two, the method comprising:
for each discrete stage of the S discrete stages, defining a representative vector for the discrete stage in a vector space defined by a set of clinical metrics based on a set of training patients labeled with the discrete stage and with values for the set of clinical metrics;
for a patient to be staged, receiving patient values for the set of clinical metrics;
generating at least one stage value for the patient to be staged based on distances in the vector space between a patient vector defined in the vector space by the patient values for the set of clinical metrics and the representative vectors for the S discrete stages in the vector space; and
displaying the at least one stage value for the patient to be staged on a display device operatively connected with the electronic processor.

2. The non-transitory computer readable medium of claim 1, wherein, for each discrete stage of the S discrete stages, the defining of the representative vector for the discrete stage includes:
defining training patient vectors in the vector space corresponding to the respective training patients labeled with the discrete stage by the values for the set of clinical metrics labeling the respective training patients; and
defining the representative vector for the discrete stage in the vector space as a centroid of the training patient vectors in the vector space.

3. The non-transitory computer readable medium of claim 1, wherein the generating of the at least one stage value includes:
identifying a closest representative vector of the representative vectors for the S discrete stages for which the distance in the vector space between the patient vector and the representative vector is shortest; and
generating a coarse stage value for the patient to be staged as the discrete stage represented by the closest representative vector, wherein the displaying includes displaying the coarse stage value.

4. The non-transitory computer readable medium of claim 1, wherein the generating of the at least one stage value includes:
identifying the two closest representative vectors of the representative vectors for the S discrete stages that are closest to the patient vector, the two closest representative vectors including a current stage representative vector corresponding to a current stage and a next stage representative vector corresponding to a next stage wherein the current stage is ordered lower than the next stage in the ordered set of S discrete stages; and
generating a fine stage value for the patient to be staged based on the current stage, the next stage, the distance in the vector space between the patient vector and the current stage representative vector, and the distance in the vector space between the current stage representative vector and the next-stage representative vector.

5. The non-transitory computer readable medium of claim 4, wherein the generating of the fine stage value is based on the current stage, the next stage, and a ratio of:
(i) a length of a projection of a current stage-to-patient vector defined as the vector starting at the current stage representative vector and ending at the patient vector onto a current stage-to-next stage vector defined as the vector starting at the current stage representative vector and ending at the next-stage representative vector, and
(ii) a length of the current stage-to-next stage vector.

6. The non-transitory computer readable medium of claim 4, wherein the generating of the fine stage value is based on the current stage, the next stage, and a ratio:
$$\frac{‖ {D}_{N \to P a} ‖}{‖ {D}_{N \to N + 1} ‖}$$
where ∥D_{N→N+1}∥ is the length of a vector D_{N→N+1} from the current stage representative vector to the next stage representative vector and D_{N→Pa} is a vector given by the dot product:
$${D}_{N \to P} \cdot \frac{{D}_{N \to N + 1}}{‖ {D}_{N \to N + 1} ‖}$$
where D_{N→P} is a vector from the current stage representative vector to the patient vector.

7. The non-transitory computer readable medium of claim 4, wherein the current stage is assigned a first integer value, the next stage is assigned a second integer value different from the first integer value, and the fine stage value is a real number lying between the first integer value and the second integer value.

8. The non-transitory computer readable medium of claim 1, wherein the distances in the vector space are computed using a distance function selected from a group including a Euclidean distance function, a Hamming distance function, a Geometric distance function, and a cosine distance function.

9. The non-transitory computer readable medium of claim 1, wherein the method further includes:
automatically assigning the discrete stage labels to the training patients using a deterministic staging algorithm based on values of a subset of the set of clinical metrics wherein the deterministic staging algorithm assigns a discrete stage selected from the predefined ordered set of S stages.

10. The non-transitory computer readable medium of claim 1, wherein the method further includes:
selecting the set of clinical metrics from a superset of clinical metrics using an automated feature selection algorithm.

11. The non-transitory computer readable medium of claim 1, wherein the method further includes associating the at least one stage value to treatment data comprising at least one intervention option to treat the patient, and the method further includes at least one of:
displaying the treatment data; and
commencing at least one treatment option to treat the patient based on the treatment data.

12. An apparatus for staging a disease having a predefined ordered set of S discrete stage where S is an integer having a value greater than or equal to two, the apparatus comprising:
at least one electronic processor; and
a non-transitory computer readable medium storing instructions readable and executable by at least one electronic processor to perform a method including:
for a patient to be staged, receiving patient values for a set of clinical metrics;
using the received patient values, defining a patient vector in a vector space defined by the set of clinical metrics;
generating at least one stage value for the patient to be staged based on distances in the vector space between the patient vector and representative vectors in the vector space that represent respective discrete stages of the predefined ordered set of S discrete stages; and
controlling a display device operatively connected with the electronic processor to display the at least one stage value for the patient to be staged.

13. The apparatus of claim 12, wherein the method further includes:
for each discrete stage of the S discrete stages, defining a representative vector for the discrete stage in a vector space defined by a set of clinical metrics based on a set of training patients labeled with the discrete stage and with values for the set of clinical metrics.

14. The apparatus of claim 13, wherein, for each discrete stage of the S discrete stages, the defining of the representative vector for the discrete stage includes:
defining training patient vectors in the vector space corresponding to the respective training patients labeled with the discrete stage by the values for the set of clinical metrics labeling the respective training patients; and
defining the representative vector for the discrete stage in the vector space as a centroid of the constructed training patient vectors in the vector space.

15. The apparatus of claim 13, wherein the generating of the at least one stage value includes:
identifying a closest representative vector of the representative vectors for the S discrete stages for which the distance in the vector space between the patient vector and the representative vector is shortest; and
generating a coarse stage value for the patient to be staged as the discrete stage represented by the closest representative vector, wherein the displaying includes displaying the coarse stage value.

16. The apparatus of claim 13, wherein the generating of the at least one stage value includes:
identifying two closest representative vectors of the representative vectors for the S discrete stages which are closest to the patient vector, the two closest representative vectors including a current stage representative vector corresponding to a current stage and a next stage representative vector corresponding to a next stage wherein the current stage is ordered lower than the next stage in the ordered set of S discrete stages; and
generating a fine stage value for the patient to be staged based on the current stage, the next stage, the distance in the vector space between the patient vector and the current stage representative vector, and the distance in the vector space between the current stage representative vector and the next-stage representative vector.

17. The apparatus of claim 16, wherein the generating of the fine stage value is based on the current stage, the next stage, and a ratio of:
(i) the length of a projection of a current stage-to-patient vector defined as the vector starting at the current stage representative vector and ending at the patient vector onto a stage-to-next stage vector defined as the vector starting at the current stage representative vector and ending at the next stage representative vector, and
(ii) the length of the stage-to-next stage vector.

18. The apparatus of claim 12, wherein the method further includes:
automatically assigning the discrete stage labels to the training patients using a deterministic staging algorithm based on values of a subset of the set of clinical metrics wherein the deterministic staging algorithm assigns a discrete stage selected from the predefined ordered set of S stages.

19. The apparatus of claim 12, wherein the method further includes:
selecting the set of clinical metrics from a superset of clinical metrics using an automated feature selection algorithm.

20. A method for staging a disease having a predefined ordered set of S discrete stages where S is an integer having a value greater than or equal to two, the method comprising:
for each discrete stage of the S discrete stages, defining a representative vector for the discrete stage in a vector space defined by a set of clinical metrics based on a set of training patients labeled with the discrete stage and with values for the set of clinical metrics by operations including:
defining training patient vectors in the vector space corresponding to the respective training patients labeled with the discrete stage by the values for the set of clinical metrics labeling the respective training patients; and
defining the representative vector for the discrete stage in the vector space as a centroid of the constructed training patient vectors in the vector space;
for a patient to be staged, receiving patient values for the set of clinical metrics;
generating at least one stage value for the patient to be staged based on distances in the vector space between a patient vector defined in the vector space by the patient values for the set of clinical metrics and the representative vectors for the S discrete stages in the vector space; and
displaying the at least one stage value for the patient to be staged on a display device.
